(19) United States
(12) Patent Application Publication
(10) Pub. No.: US 2024/0335599 A1
(43) Pub. Date: Oct. 10, 2024

(54) VACUUM PORT CONNECTION FOR MEDICAL FLUID DRAINAGE SYSTEM

(71) Applicant: C.R. Bard, Inc., Murray Hill, NJ (US)

(72) Inventors: Jason Jishen Cheng, Covington, GA (US); David Simiele, Roswell, GA (US)

(21) Appl. No.: 18/034,943

(22) PCT Filed: Nov. 2, 2021
(86) PCT No.: PCT/US2021/057760

§ 371 Date: May 2, 2023

Related U.S. Application Data
(60) Provisional application No. 63/109,093, filed on Nov. 3, 2020.

(51) Int. Cl. A61M 1/00

(52) U.S. Cl. CPC A61M 1/72; A61M 1/784; A61M 2205/7536

(57) ABSTRACT

An embodiment of a medical fluid drainage system includes a drainage container configured to receive and hold a fluid from a patient. The drainage container is fluidly coupled and reversibly attached to a drainage bag, which receives some of fluid initially held by the drainage container. A vacuum port is provided in the drainage container for attachment to a negative pressure source. The vacuum port may be formed in a removable lid of the drainage container. The negative pressure source may remove excess air from the drainage container and drainage bag that can inflate the drainage bag and prevent fluid flow thereto.

### CROSS-REFERENCE TO RELATED APPLICATIONS

This application claims priority to U.S. Provisional Patent Application No. 63/109,093 filed on Nov. 3, 2020, the disclosure of which is incorporated herein, in its entirety, by this reference.

### BACKGROUND

There is often a medical need to drain fluid from patients. For example, urine

may be drained from a patient's bladder for a variety of reasons (e.g., unconsciousness, incontinence, etc.). In another example, a patient recovering from thoracic surgery (e.g., heart or lung surgery) may require a chest drainage to remove pleural effusion from the chest cavity. In order to drain fluid from hollow organs (such as the bladder) or body cavities, particularly over long periods of time, drainage systems are commonly used. In any drainage system a drainage tube is used to collect fluid from the hollow organ or body cavity and transport the fluid to a collection device such as a bag or container, which received and stores the fluid.

In practice, a first end of a drainage tube such as a catheter, a Foley urinary catheter, or other type of medical tubing, may be positioned in the hollow organ or body cavity. A second end of the drainage tube may be releasably connected to the collection device. The collection device is often positioned at a level below the point of drainage from the patient's body. For example, if the patient is primarily resting in a bed, the collection device may be placed lower than the patient, e.g., under the bed or on the floor. By placing the collection device lower than the patient, a siphon may be created by harnessing gravity to assist the drainage of fluid.

### SUMMARY

Embodiments disclosed herein are directed to medical fluid drainage systems configured to facilitate siphoning and disposal of a fluid collected from a patient and methods of using such medical fluid drainage systems. In an embodiment, a drainage container is provided for use in a medical fluid drainage system. The drainage container may be formed by one or more walls that define a reservoir for holding a fluid therein. An inlet port may be defined within one of the one or more walls and configured to fluidly couple to a drainage catheter. An outlet port may be defined within one of the one or more walls and configured to be fluidly coupled to a drainage bag. A vacuum port may be defined within one of the plurality of walls and configured to attach to a negative pressure source. One of the one or more walls may be a lid forming a top surface of the drainage container and the vacuum port may be defined within the lid. The inlet port may also be defined within the lid. At least some of the one or more walls may define an upper opening in the drainage container and the lid may removably attached to the drainage container to seat upon and cover the upper opening. The lid may be configured to form a fluid tight seal with edges of the one or more walls defining the upper opening. The lid may further include a vent aperture covered by a gas-permeable, liquid-impermeable membrane. Another gas-permeable, liquid-impermeable membrane may be positioned to extend across a lumen of the vacuum port. In some embodiments, the membrane may be positioned on a bottom surface of the lid. In some embodiments, the vacuum port defines an annular wall that extends beyond an outer surface of the one of the plurality of walls and is formed with a locking connection structure configured to connect with an opposing connector attached to the negative pressure source.

In another embodiment, a medical fluid drainage system is disclosed. The medical fluid drainage system may include a number of tubes, for example, a drainage tube, an evacuation tube, and a transfer tube. The drainage tube may have a first end configured for placement within a hollow organ or body cavity of a patient and a second end. The evacuation tube may have a first end configured to attach to a negative pressure source and a second end. The transfer tube may have a first end and a second end. The medical fluid drainage system may further include a drainage container that defines a reservoir for holding a fluid therein. An inlet port may be defined within a first portion of the drainage container and configured to attach to the second end of the drainage catheter. An outlet port may be defined within a second portion of the drainage container and configured to attach to the first end of the transfer tube. A vacuum port may be defined within a third portion of the drainage container and configured to attach to the second end of the evacuation tube. The system may further include a drainage bag with an inlet port configured to attach to the second end of the transfer tube. A negative pressure source may be coupled to the vacuum port via the evacuation tube and configured to pull air from the drainage container to maintain a pressure equilibrium within the drainage container and the drainage bag. A positive pressure source may be coupled with the drainage tube and configured to provide air flow through the drainage tube and into the drainage container. The drainage container may further have a removable lid and the third portion of the drainage container defining the vacuum port may form a portion of the removable lid. The first portion of the drainage container defining the inlet port may form another portion of the removable lid. A gas-permeable, liquid-impermeable membrane may be positioned to extend across a lumen of the vacuum port. The membrane may be positioned on a bottom surface of the removable lid. The lid may further include a vent opening covered by a gas-permeable, liquid-impermeable membrane.

In a further embodiment, a method of providing medical fluid drainage to a patient is disclosed. A bodily fluid from a patient may be received into a drainage container through a drainage tube. At least a portion of the bodily fluid may be transferred from the drainage container to a drainage bag fluidly connected to the drainage container. Excess air may be removed from and pressure may be reduced within the drainage container using a negative pressure source connected to the drainage container. Excess air may be removed from the drainage bag via the fluid connection with the drainage container to provide an equilibrium pressure in the drainage bag. A positive pressure air flow may be flowed into the drainage tube thereby flow into the drainage container. The drainage container may include a removable lid defining a vacuum port connected to the negative pressure source and the removal of excess air from the drainage container may include flowing air out of the drainage container through the vacuum port.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Features from any of the disclosed embodiments may be used in combination with one another, without limitation. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a schematic elevation view of a general embodiment of a medical fluid drainage system.

FIG. 2 is an elevation view of an embodiment of a drainage container for a medical fluid drainage system.

FIG. 3A is a front elevation view of an embodiment of a lid for a drainage container for a medical fluid drainage system.

FIG. 3B is a top, front isometric view of the embodiment of the lid of FIG. 3A.

FIG. 3C is a bottom, rear isometric view of the embodiment of the lid of FIG. 3A.

FIG. 4A is a top, front isometric view of an embodiment of a lid for a drainage container for a medical fluid drainage system.

FIG. 4B is a bottom, rear isometric view of the embodiment of the lid of FIG. 4A.

FIG. 5 is a flow diagram of a method of operating a medical fluid drainage system with a vacuum port connection.

### DETAILED DESCRIPTION

Embodiments disclosed herein are directed to medical fluid drainage systems configured to facilitate siphoning and disposal of a fluid collected from a patient and methods of using such medical fluid drainage systems. In particular, a medical fluid drainage system is designed to use airflow to improve fluid clearance from drainage tubing, including clearing fluid from dependent loops in the drainage tube. For example, any embodiment of the medical fluid drainage system may include a drainage container configured to couple with a drainage tube to receive and hold a fluid from a patient. The drainage container may also be fluidly coupled to and reversibly attached to a drainage bag. The drainage bag may be configured to receive at least some of the fluid initially held by the drainage container. The bag may be detached from the drainage container and disposed of and replaced by an empty drainage bag.

The drainage bag systems disclosed herein may be configured to be used in any system that drains one or more fluids from a patient. For example, the drainage bag systems disclosed herein may be used in a urinary drainage system (e.g., a Foley catheter and drainage system). As such, the drainage bag systems disclosed herein may be configured to be a urinary drainage bag system. In other embodiments, the drainage bag systems disclosed herein may be used in a blood drainage system, a pleural drainage system, a peritoneal drainage system, a bowel drainage system (e.g., a stool collection bag), or another other suitable drainage system.

Dependent loops in tubing of drainage systems form when a section of the drainage tubing is lower than the rest of the system. For example, if the outlet of the drainage tube is connected at a top portion of the collection device, the outlet may be above the floor. Often the drainage tube is rather long, e.g., 5 feet long or more, and a midsection of the drainage tube will droop downward and rest upon the floor and then rise upward again to where it connects to the collection devices. A dependent loop is thus formed by excess drainage tubing in the drainage system where liquid can accumulate. Dependent loops can trap fluid and impede drainage, thereby reducing the overall drainage of fluid from the patient. This increases the residual volume of retained fluid in the hollow organ or body cavity and can result in a negative impact on patient outcome. Residual fluid that collects in the dependent loop may also pose a bacterial growth risk.

One method to clear dependent loops is to apply a positive or negative pressure to push or pull the residual urine into the collection device. Typical drainage systems use constant pressure and low airflow to move the fluid in the dependent loop. However, significant amounts of residual fluid are often left in the tube. In order to maximize clearance of fluid from the drainage tube to the collection device, a higher airflow through the tube is required. However, a higher airflow creates a higher risk of inflating a drainage bag, which is typically either the only element of the collection device or the final repository in the drainage system. Inflation of the drainage bag prevents fluid from flowing into the bag as there is nowhere to displace the air.

In one or more embodiments disclosed herein, a vacuum port may be provided on the drainage container and is configured for connection with a negative pressure source. For example, an evacuation tube may be attached to the vacuum port at a first end and to a negative pressure source at a second end. As the drainage container is in fluid communication with the drainage bag, the negative pressure source is able to remove any excess air that may collect in and inflate the drainage bag. Removing excess air from the drainage system allows the drainage system to maintain a pressure and air-volume equilibrium and prevent or reduce inflation of the drainage bag. This allows the drainage bag to properly fill while also allowing for a high relative air flow in the system to prevent fluid pooling in dependent loops of the drainage tube.

By adding a vacuum port to the drainage container in a fluid drainage system, excess air can be removed from the drainage container and the drainage bag. In one or more embodiments, the vacuum port may be provided in a removable lid that covers a top opening in the drainage container in a fluid-tight manner. An evacuation tube may be attached to the vacuum port at a first end and to a negative pressure source at a second end. The evacuation tube may be fitted with a quick connector allows for simple attachment and detachment of a vacuum line to the negative pressure source to remove excess air from the drainage container and drainage bag. The connector feature design may be, but is not limited to, common medical connector types (e.g., Luer, ENFit) or proprietary connector types. Removing excess air allows the system to be at a pressure and air-volume equilibrium to prevent bag inflation. A porous, air permeable, liquid impermeable membrane may be positioned underneath the vacuum port across the lumen thereof to maintain the integrity of the closed drainage system and the negative pressure source. The membrane may filter air and prevent ingress of outside contaminants to the closed system, as well as preventing collected bodily fluids from leaking to the outside from the vacuum port.

The addition of a vacuum port to the drainage container enables high air flow rate clearance mechanisms attached to the drainage tube to maximize the amount of bodily fluid cleared from dependent loops in the drainage tube. High air flow is often necessary to clear fluid pooling in low loops of long drainage tubes. However, high air flow through the drainage hose introduces high air pressure in the drainage container and often pushes air into the drainage bag. If the drainage bag inflates with air, fluid in the drainage bag is displaced and additional fluid will not flow into the drainage bag. By providing a negative pressure to the drainage container through the vacuum port, excess air entering from the drainage tube is evacuated, thereby preventing pressure build-up in the drainage container and ultimately in the drainage bag. Removal of excess pressure in the drainage system prevents bag inflation and allows desired fluid flow into the drainage bag. By maintaining a pressure equilibrium in the drainage bag, high flow-rate clearance mechanisms provided in the drainage tube are able to be effectively used to maximize clearance of residual fluid in dependent loops.

In any embodiment, a filter or membrane may be positioned at the interface between the vacuum port and a body of the drainage container across a lumen defined by the vacuum port. The membrane may be a porous, gas-permeable, but liquid-impermeable material. The membrane may filter air and prevent ingress of outside contaminants to maintain the integrity of the closed drainage system. The membrane also prevents or limits fluid from leaking out of the drainage container through the vacuum port, for example, in an instance where the drainage container is knocked over from a normal, upright position, and particularly when an evacuation tube is not connected to the vacuum port.

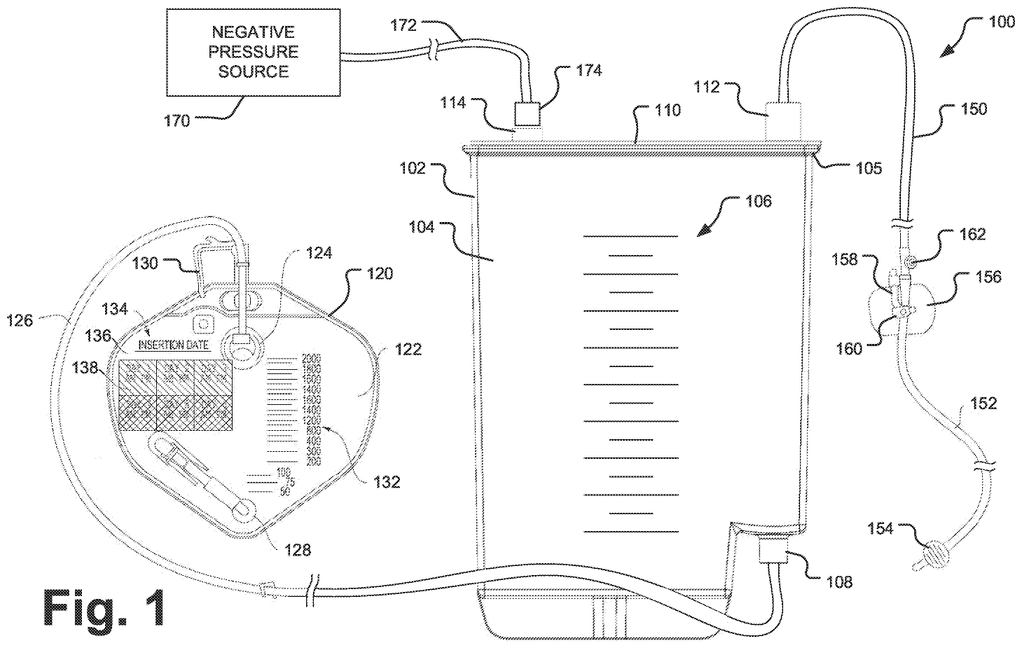

FIG. 1 is a schematic front elevation view of a medical fluid drainage system 100, and is a general example of any embodiment. The drainage system 100 includes a drainage container 102 that defines an interior reservoir 104 configured to receive and hold a fluid from a patient therein. The drainage container 102 may be defined by a number of side walls extending from a base wall, e.g., shaped generally as a hollow cuboid or rectangular prism as shown. In other embodiments, the drainage container 102 may be formed as a hollow cylinder or any other hollow three dimensional shape that can define an interior reservoir 104. A container outlet port 108 may be formed in a bottom wall or lower portion of the drainage container 102. In any embodiment, the drainage container may define an opening in a top portion thereof bounded by a container rim 105. In such embodiments, a removable lid 110 may be provided to cover the opening in the top of the drainage container 102.

The drainage container 102 may be formed as a single piece, e.g., as a molded plastic unit, in order to form a fluid-tight volume defining the interior reservoir 104. In some embodiments, the drainage container 102 may be formed of polyvinyl chloride (“PVC”), a di-2-ethylhexyl phthalate (“DEHP”) free polymer (e.g., DEHP-free PVC), or any other suitable material. The drainage container may be rigid, semi-rigid, flexible, or resilient, or a combination of any of these attributes depending upon the type of material or materials used in the formation of the walls of the drainage container 102. In some embodiments, the drainage container 102 may be formed by a number of separate walls or panels that are assembled together to define the interior reservoir 104 and that are connected together via fluid tight bonds or seals (e.g., by welds) to prevent leakage of a fluid from the drainage container 102. In some embodiments, the interior volume 104 may be divided into two or more separate compartments (not shown). Such compartments may be fluidly connected, for example, by overflow of the fluid over a wall dividing compartments once a first compartment is filled.

A volume of fluid collected in the interior reservoir 104 may be measured by a plurality of graduated marks 106 provided on a wall of the drainage container 102. The graduated marks 106 may facilitate determination of a volume of fluid discharged by a patient during a time span. At least a portion of one of the walls defining the drainage container 102 may be formed from one or more materials that are transparent and/or translucent, thereby permitting a user of the drainage system 100 to determine a volume of fluid within the interior reservoir 104. The graduation marks may be superimposed on the transparent or translucent area of the drainage container 102. In some embodiments the drainage container 102 may be made uniformly of the same material such that the entire drainage container 102 is transparent or translucent. In other embodiments, the various surfaces or walls of the drainage container 102 may be made of different materials such that some surfaces are transparent while other surfaces are opaque.

The lid 110 may define a number of ports therein. A drainage tube port 112 may be formed in a portion of the lid 110 to allow deposition of a fluid from a patient into the interior reservoir 104 of the drainage container 102. A drainage tube 150 from a patient may connect to the drainage tube port 112 via any common connection method, e.g., a Luer lock connector, and ENFit connector, a valved connector, any proprietary connector design, or even a friction fit between the drainage tube 150 and the drainage tube port 112. In any embodiment, the drainage tube port 112 may include an anti-reflux valve (not shown) to prevent fluid from flowing in a reverse direction out of the drainage container 102 into the drainage tube 150. The drainage tube 150 may be made out of any typical medical grade plastic tubing material, e.g., polyvinyl chloride (PVC), polyethylene, thermoplastic elastomers (TPE), nylon, or silicone.

The drainage tube 150 may connect to a patient drainage line, an embodiment of which is depicted in FIG. 1. A distal end of the drainage tube 150 may connect to a drainage catheter 152 or other patient drainage conduit via a sample port 162. The sample port 162 and other access ports or valves may be mounted on an adhesive patch 156 that may be affixed to the skin of a patient for ease of location and access of such ports and valves by medical personnel, to minimize movement of the drainage catheter 152, and to reduce or prevent strain on the drainage catheter 152 that could be caused by movement of the connected drainage tube 150 (e.g., when the patient moves in the bed or is ambulatory or if the drainage container 102 is moved).

The drainage catheter 152, as shown in FIG. 1, may be a Foley-type catheter with a retention balloon 154 at a distal end thereof. The balloon is shown in an inflated state in FIG. 1. As is well known, when the distal end of a Foley-type catheter is placed within a hollow organ or body cavity, the retention balloon 154 may be inflated with air. The large size of the retention balloon resists or prevents the drainage catheter 152 from sliding out or accidentally being pulled out of a hollow organ or body cavity. The Foley-type catheter 152 may include a separate air tube along its length that terminates at the retention balloon 154 to fill or evacuate the retention balloon 154. A balloon port 158 is provided at the proximal end of the Foley-type catheter in order to fill or evacuate the retention balloon 154 with air, e.g., by connecting a syringe filled with air to the balloon port 158. A valve 160 may also be provided on the proximal end of the Foley-type catheter to open and close the separate air tube. While a Foley-type catheter is shown in FIG. 1 as an example of a drainage catheter 152, any type of catheter or other drainage tubing may be used and placed inside a hollow organ or body cavity of a patient for drainage of fluid. In some embodiments, the drainage tube 150 may extend directly from a hollow organ or body cavity of a patient to the drainage tube port 112 of the drainage container 102.

A vacuum port 114 may be defined in another portion of the lid 110. An evacuation tube 172 may connect to the vacuum port 112 via a vacuum port connector 174 affixed to a first end of the evacuation tube 172. The vacuum port connector 174 may be provided as any common tube connection device, e.g., a Luer lock connector, and ENFit connector, a valved connector, any proprietary connector design, or even a friction fit between the evacuation tube 172 and the vacuum port 114. The evacuation tube 172 may be made out of any typical medical grade plastic tubing material, e.g., polyvinyl chloride (PVC), polyethylene, thermoplastic elastomers (TPE), nylon, or silicone. The connection between the evacuation tube 172 and the vacuum port 114 may be a gas-tight connection requiring appropriate seals between surfaces of the vacuum port 112 and the vacuum port connector 174.

The second end of the evacuation tube 172 may be connected to a negative pressure source 170 that provides at least a slight vacuum suction or negative pressure to the drainage container 102 as further described below. A connector (not shown) may be provided on a second end of the evacuation tube 172 to connect the evacuation tube 172 to the negative pressure source 170. This connector may be provided as any common tube connection device, e.g., a Luer lock connector, and ENFit connector, a valved connector, any proprietary connector design, or even a friction fit between the evacuation tube 172 and a port on the negative pressure source 170. The connection between the evacuation tube 172 and the negative pressure source 170 may be a gas-tight connection requiring appropriate seals between surfaces of the port on the negative pressure source 170 and the connector on the second end of the evacuation tube 172.

The container outlet port 108 may be formed in a wall of the drainage container 102 in a lower portion or the bottom of the drainage container 102. The drainage container 102 may be fluidly coupled to and reversibly attached to (e.g., may be attached to and detached from) a drainage bag 120 via a transfer tube 126 connected to the container outlet port 108. The transfer tube 126 may be made out of any typical medical grade plastic tubing material, e.g., polyvinyl chloride (PVC), polyethylene, thermoplastic elastomers (TPE), nylon, or silicone. A first end of the transfer tube 126 may have a connector configured to be attached to and removed from the container outlet port 108. The connector on the first end of the transfer tube 126 may be provided as any common tube connection device, e.g., a Luer lock connector, and ENFit connector, a valved connector, any proprietary connector design, or even a friction fit between the transfer tube 126 and the container outlet port 114. The connection between the transfer tube 126 and the container outlet port 108 may be a fluid-tight connection requiring appropriate seals between surfaces of the container outlet port 108 and the connector on the first end of the transfer tube 126.

A second end of the transfer tube 126 connects to the drainage bag 120 at a bag inlet port 124. In some embodiments, the transfer tube 126 is permanently connected to and hermetically sealed with the drainage bag 120 at the bag inlet port 124. In other embodiments, the transfer tube 126 may be removably connected to the drainage bag 120 via a fluid-tight connection. The transfer tube 126 thus transports patient fluid from the drainage container 102 to the drainage bag 120 for final collection, handling, and disposal as necessary. The drainage bag 120 may further include a bag outlet port 128 positioned at or near a bottom portion of the drainage bag 120. The bag outlet port 128 may be configured to allow a fluid collected in the drainage bag 120 to flow or drain from the drainage bag 120 (e.g., for collection or extraction of the fluid from the drainage bag 120). In one example implementation, the bag outlet port 128 may include a Safety-Flow™ outlet device or another similar outlet device.

The drainage bag 120 defines an interior volume configured to receive and hold at least a portion of the fluid previously held in and transferred from the interior space 106 of the drainage container 102. The drainage bag 120 may be made from any typical medical grade plastic material, e.g., polyvinyl chloride (PVC), polyethylene, thermoplastic elastomers (TPE), nylon, or silicone. In some embodiments, the drainage bag 120 may be made by bonding or adhering together two flat pieces of medical grade plastic about their perimeters, for example, by lamination, ultrasonic welding, chemical welding, or other methodology to create a fluid tight seal around the perimeter and thus form the interior volume when a space between the two flat pieces of plastic is expanded.

A front panel 122 of the drainage bag 102 may further include one or more graduated markings 132 that may indicate an amount of a fluid collected in the drainage bag 120. For example, the graduated markings 132 may facilitate determination of the amount of fluid discharged by the patient in a time span (e.g., a predetermined time span). In some embodiments, the graduated markings 132 may facilitate determining or approximating a time and/or date for draining, removing, and/or changing out the drainage bag 120.

An indicator panel 134 may also be provided on the front panel 122 of the drainage bag 120. The indicator panel 134 may be configured to allow for indication of completion of a task (e.g., a patient care protocol) related to the drainage system 100 for patient charting and compliance purposes. For example, a patient data section 136 may provide information related to the drainage bag 120 and/or patient care including information related to the patient (e.g., the patient's name, the procedure performed on the patient, etc.), information related to the caregiver (e.g., the responsible physician's name, the responsible nurse's name, etc.), the drainage tube insertion date, etc. Further, a tracking section 138 may provide calendar and time tracking forms that can be marked on the front panel to record, for example, fluid volume levels at specific dates and times, protocol completion at specific dates and times, etc.

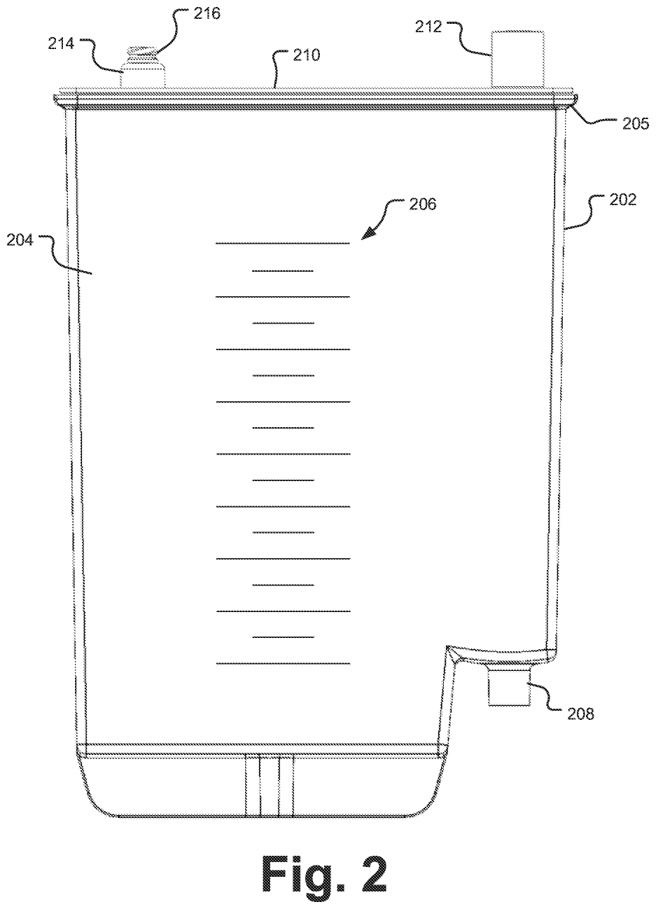

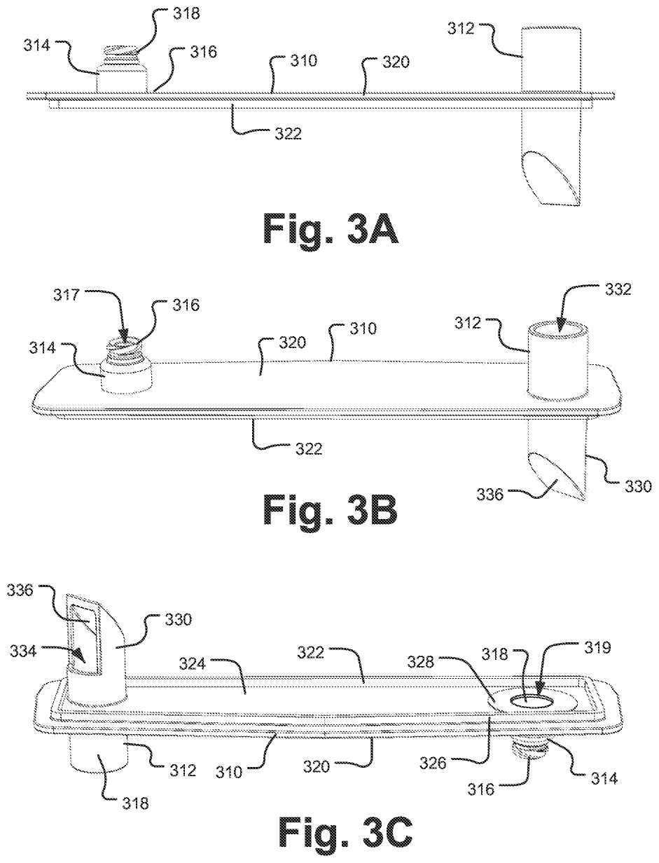

An embodiment of a lid 210 for the drainage container 202 is depicted in FIGS. 2-3C. In FIG. 2, the lid 210 is shown in place on the top of the drainage container 202 against the container rim 205 and covering the interior reservoir 204. Graduation marks 206 and the container outlet port are further depicted in FIG. 2. As in FIG. 1, the lid may include a drainage tube port 212 formed in a portion of the lid 210 to allow deposition of a fluid from a patient into the interior reservoir 204 of the drainage container 202. A drainage tube from a patient may connect to the drainage tube port 212 via any common connection structure (e.g., a Luer lock connector, an ENFit connector, a valved connector, or any proprietary connector design), or even a friction fit between the drainage tube and the drainage tube port 212. In any embodiment, the drainage tube port 212 may include an anti-reflux valve (not shown) to prevent fluid from flowing in a reverse direction out of the drainage container 202 into a drainage tube. The lid 210 may further include a vacuum port 214 formed in a portion of the lid 210. An outward extending end of the vacuum port 214 may be formed with a connection structure 216 for coupling with a connector on an evacuation hose (e.g., a Luer lock connector, an ENFit connector, a valved connector, any proprietary connector design).

The lid 310 of the example embodiment of FIG. 2 is shown in isolation and greater detail in FIGS. 3A-3C. The lid 310 may be defined by a top surface 320 and a bottom surface 324. A lip 322 may extend outward from and normal to the bottom surface 324 of the lid 310 as a short continuous wall that defines a closed perimeter. The lip 310 may be sized and shaped to fit within and conform to the opening in the top of the drainage container defined by the rim. A portion of the bottom surface 324 may extend beyond the perimeter defined by the lip 322 to form a flange 325 that seats on top of the rim of the drainage container. A seal 326 may be provided on the outer edge of the lip 322 to form a fluid-tight seal with the drainage container when the lid 310 is placed on the rim of the drainage container. The seal 326 may be in the form of a gasket of an elastomeric material (e.g., a rubber or silicone) that is configured for removable connection of the lid 310 to the drainage container to effectively seal the opening in the top of the drainage container defined by the rim.

As noted, the lid 310 may include a drainage tube port 312 and a vacuum port 314. The drainage tube port 312 may extend above the top surface 320 of the lid 310 and below the bottom surface 324 of the lid 310. The drainage tube port 312 may define a port inlet 332 above the top surface 324 configured to connect to drainage tube to allow flow of a fluid from a patient into the interior reservoir of the drainage container. A drainage tube from a patient may connect to the drainage tube port 312 via any common connection method, e.g., a Luer lock connector, and ENFit connector, a valved connector, any proprietary connector design, or even a friction fit between the drainage tube and the drainage tube port 312. In any embodiment, the drainage tube port 312 may include an anti-reflux valve (not shown) to prevent fluid from flowing in a reverse direction out of the drainage container into the drainage tube.

The drainage tube port 312 may extend below the bottom surface 324 of the lid 310 into interior reservoir of the drainage container in the form of a flow director conduit 330. The flow director conduit 330 may define a port outlet 334 in a sidewall thereof. An end surface 336 of the flow director conduit 330 may be formed at an angle with respect to the sidewall of the flow director conduit 330 to direct fluid flowing through the drainage tube port 312 through the port outlet 334 in the sidewall to prevent pooling of fluid in the drainage tube port 312. The design of the flow director conduit 330 may be helpful in directing fluid into the drainage container. For example, if the drainage container is composed of two or more compartments as described above, the port outlet 334 of the flow director conduit 330 may be oriented to ensure that the fluid flowing through the drainage tube port 312 is only deposited in a desired one of the compartments in the drainage container.

As shown in the embodiment presented in FIGS. 3A-3C, the vacuum port 314 may be positioned on another portion of the lid 310 ad extend upward from the top surface 320 of the lid 310. A locking structure 316 may be provided at the port outlet 317 of the vacuum port 314 and configured to connect to a first end of an evacuation tube. (The second end of the evacuation tube is connected to a negative pressure source). The locking structure 316 may be formed as any common tube connection structure, e.g., a Luer lock connector (in the depicted embodiment, a male Luer connector), a valved connector, or following any proprietary connector design. In the depicted embodiment, a female Luer connector may be provided on the end of the evacuation tube for connection with the locking structure 316. In other embodiments, the locking structure may be a female component at the connector on the end of the evacuation tube may be a male component. The connection between the evacuation tube and locking structure 316 on the vacuum port 314 may be a gas-tight connection requiring appropriate seals between surfaces of the vacuum port 312, the locking structure 316, and a vacuum port connector on the first end of the evacuation tube.

A membrane 318 may be positioned across the lumen of the vacuum port 314. In the implementation shown in FIG. 3C, the membrane 318 may be positioned against the bottom surface 324 of the lid 310 over the vacuum port inlet 319. The membrane may be held in place by a retention ring 328 affixed to the bottom surface 324 of the lid 310. In some embodiments, a perimeter edge of the membrane 318 may be sandwiched between the retention ring 328 and the bottom surface 324 of the lid 310. In other embodiments, the retention ring 328 may encapsulate a perimeter edge of the membrane 318, e.g., through a molding process, such that the retention ring 328 and the membrane 318 form a single unit. A surface of the retention ring 328 may be adhered, welded (e.g., by sonic welding) or otherwise affixed to the bottom surface 324 of the lid 310 in a position such that the membrane 318 spans across the lumen of the vacuum port 314. The membrane 118 may be made of a gas-permeable, liquid impermeable material such as Gore-Tex® or other similar fabric in order to prevent fluid from the drainage container from leaking out of the vacuum port 314 (e.g., should the drainage container be knocked over), while allowing air to flow through the vacuum port 314 from the interior reservoir of the drainage container.

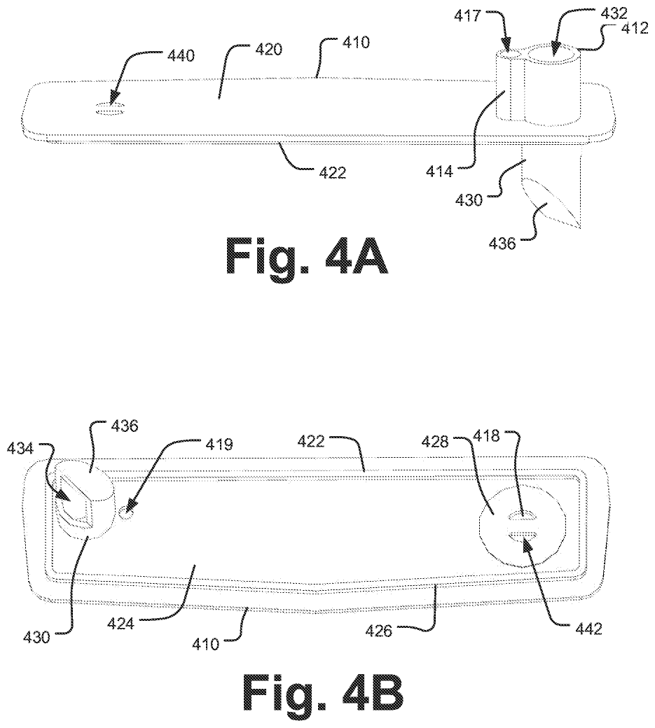

An alternative implementation of a lid 410 for a drainage container is shown in detail in FIGS. 4A and 4B. The lid 410 may be defined by a top surface 420 and a bottom surface 424. A lip 422 may extend outward from and normal to the bottom surface 424 of the lid 410 as a short continuous wall that defines a closed perimeter. The lip 410 may be sized and shaped to fit within and conform to the opening in the top of the drainage container defined by the rim. A portion of the bottom surface 424 may extend beyond the perimeter defined by the lip 422 to form a flange 425 that seats on top of the rim of the drainage container. A seal 426 may be provided on the outer edge of the lip 422 to form a fluid-tight seal with the drainage container when the lid 410 is placed on the rim of the drainage container. The seal 426 may be in the form of a gasket of an elastomeric material (e.g., a rubber or silicone) that is configured for removable connection of the lid 410 to the drainage container to effectively seal the opening in the top of the drainage container defined by the rim.

As in any embodiment, the lid 410 may include a drainage tube port 412 and a vacuum port 414. The drainage tube port 412 may extend above the top surface 420 of the lid 410 and below the bottom surface 424 of the lid 410. The drainage tube port 412 may define a port inlet 432 above the top surface 424 configured to connect to drainage tube to allow flow of a fluid from a patient into the interior reservoir of the drainage container. A drainage tube from a patient may connect to the drainage tube port 412 via any common connection method, e.g., a Luer lock connector, an ENFit connector, a valved connector, any proprietary connector design, or even a friction fit between the drainage tube and the drainage tube port 412. In any embodiment, the drainage tube port 412 may include an anti-reflux valve (not shown) to prevent fluid from flowing in a reverse direction out of the drainage container into the drainage tube.

The drainage tube port 412 may extend below the bottom surface 424 of the lid 410 into interior reservoir of the drainage container in the form of a flow director conduit 430. The flow director conduit 430 may define a port outlet 434 in a sidewall thereof. An end surface 436 of the flow director conduit 430 may be formed at an angle with respect to the sidewall of the flow director conduit 430 to direct fluid flowing through the drainage tube port 412 through the port outlet 434 in the sidewall to prevent pooling of fluid in the drainage tube port 412. The design of the flow director conduit 430 may be helpful in directing fluid into the drainage container. For example, if the drainage container is composed of two or more compartments as described above, the port outlet 434 of the flow director conduit 430 may be oriented to ensure that the fluid flowing through the drainage tube port 412 is only deposited in a desired one of the compartments in the drainage container.

In the embodiment presented in FIGS. 4A and 4B, the vacuum port 414 may be positioned immediately adjacent to the drainage tube port 412 in the lid 410 and extend upward from the top surface 420 of the lid 410. In this implementation, the vacuum port 414 may be formed in conjunction with the drainage tube port 412 as a singular structure. Further in this implementation, a first end of an evacuation tube may be connected to the vacuum port 414, for example, with a barbed connector (not shown) inserted into the vacuum port outlet 417. Other connection structures may be provided at the port outlet 317 of the vacuum port 414 and configured to connect with the first end of an evacuation tube. As noted above, the second end of the evacuation tube may be configured to connect to a negative pressure source). In some implementations, a common connector structure may be formed to connect to both the drainage tube and the evacuation tube on one side and to both the drainage tube port and the vacuum port, respectively, on the other side of the connector. The connection between the evacuation tube and the vacuum port 414 may be a gas-tight connection requiring appropriate seals between surfaces of the vacuum port 412 and any connection structure on the first end of the evacuation tube.

In the implementation depicted in FIGS. 4A and 4B, the port inlet 419 to the vacuum port 414 may be formed as merely an aperture in the lid 410. In some implementations the port inlet 419 for the vacuum port may be covered by a gas permeable, fluid impermeable membrane (not shown).

Further, as shown in FIGS. 4A and 4B, any embodiment of the lid, for example, as depicted in the lid 410, one or more vent apertures 440 may be provided. The vent apertures 440 may provide additional air flow through the drainage system when negative pressure is applied through the vacuum port and thereby moderate the force of the negative pressure on the drainage bag. In order to prevent fluid from the drainage container from leaking out of the vent apertures 440 (e.g., should the drainage container be knocked over), while allowing air to flow through the vent apertures 440 into the interior reservoir of the drainage container, a gas-permeable, liquid-impermeable membrane 418 may be positioned on the bottom surface 424 of the lid 410 to cover the vent apertures 440. The membrane 418 may be made of a gas-permeable, liquid-impermeable material such as Gore-Tex® or other similar fabric.

The membrane 418 may be held in place by a retention plate 428 affixed to the bottom surface 424 of the lid 410. The retention plate 428 may define plate apertures 442 of the same or similar size as the vent apertures 440. The plate apertures 442 may be positioned in alignment with the vent apertures 440 in order to allow air flow through the lid 410 into the drainage container. In other embodiments, the retention plate 428 may have a single aperture (similar to the retention ring of FIG. 3C) sized to cover all of the vent apertures 440. This form may provide for easier construction of the retention plate 428 while allowing for volume control of air flow through the size and number of vent apertures 440 in the lid 410. In some embodiments, a perimeter edge of the membrane 418 may be sandwiched between the retention plate 428 and the bottom surface 424 of the lid 410. In other embodiments, the retention plate 428 may encapsulate a perimeter edge of the membrane 418, e.g., through a molding process, such that the retention plate 428 and the membrane 418 form a single unit. A surface of the retention plate 428 may be adhered, welded (e.g., by sonic welding) or otherwise affixed to the bottom surface 424 of the lid 410 in a position such that the membrane 418 spans across the lumen of the vent apertures 440.

Other implementations of the vacuum port are contemplated. For example, the vacuum port may be positioned in any location or portion of the lid. In one contemplated implementation, the vacuum port may be formed concentrically with the drainage tube port on the lid, forming a sleeve about the drainage tube port. In such an implementation, a common connector could be used to attach both the fluid drainage tube and the evacuation tube at a single point, providing for ease of connection of tubing to the drainage container. Such an implementation may provide an improvement in tubing management if the fluid drainage tube and the evacuation tube are concentric for at least a length of tubing run to a manifold or other common location before splitting to separate tubing.

A membrane 318 may be positioned across the lumen of the vacuum port 314. In the implementation shown in FIG. 3C, the membrane 318 may be positioned against the bottom surface 324 of the lid 310 over the vacuum port inlet 319. The membrane may be held in place by a retention ring 328 affixed to the bottom surface 324 of the lid 310.

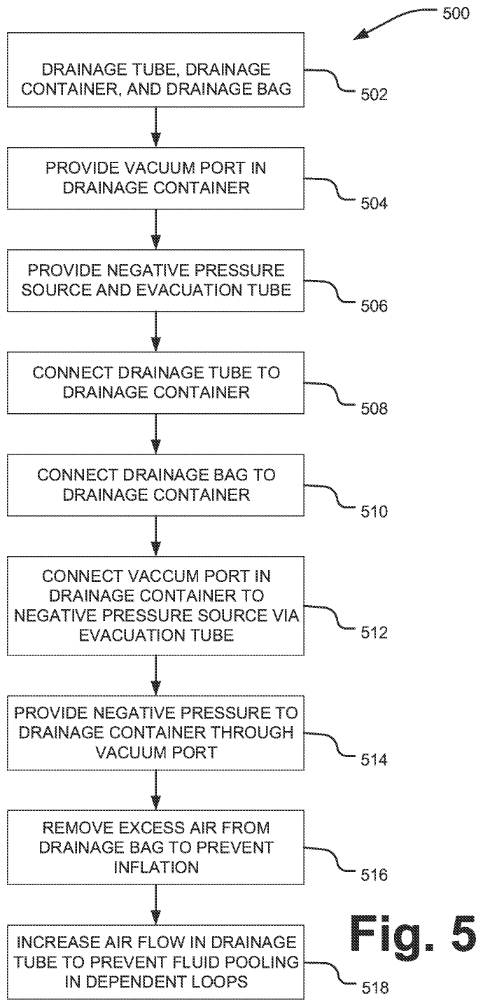

A method 500 of using implementations of medical fluid drainage systems as described herein is presented in FIG. 5. As a first step 502, a patient is provided with a medical fluid drainage system including a drainage tube, a drainage container and a drainage bag. In addition to these basic elements for a medical fluid drainage system, in a second step 504, a vacuum port is provided in the drainage container. In embodiments disclosed herein, the vacuum port may be provided in a lid to the drainage container that seals a top opening of the drainage container. In a further related step 506, a negative pressure source and an evacuation tube may be provided to further augment the standard drainage system.

With all of the components of a medical fluid drainage system with the addition of a negative pressure component available, the clinician may place the drainage tube in a hollow organ or body cavity of a patient and connect the drainage tube to the drainage container as indicated in step 508. The drainage bag is also connected to the drainage container as indicated in step 510. Further, a vacuum port provided in the drainage container, for example in the lid of the drainage container, is connected to a negative pressure source with an evacuation tube as indicate din step 512.

Now that the medical fluid drainage system is set up, the clinician can activate the negative pressure source and provide negative pressure to the drainage container through the vacuum port as indicated in step 514. The negative pressure source may thereby effectively remove excess air from the drainage bag and prevent inflation of the drainage bag as indicated in step 516. With constant or scheduled removal of excess air from the drainage container and further from the drainage bag, increased air flow through the drainage tube can be provided to reduce pooling of fluid in dependent loops of the drainage tube and provide more effective patient treatment as indicated in step 518. For example, a positive air input could provide through the sample port at the distal end of the drainage tube of sufficient pressure and flow to push the fluid through the drainage tube, even through dependent loops. The negative pressure source may thus operate to alleviate any excess air pressure build up in the drainage container due to increased air flow through the drainage tube and further prevent inflation of the drainage bag that may block filling of the drainage bag with fluid from the patient.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the structures disclosed herein, and do not create limitations, particularly as to the position, orientation, or use of such structures. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The drawings are for purposes of illustration only and the dimensions, positions, order and relative sizes reflected in the drawings attached hereto may vary.

The above specification, examples, and data provide a complete description of the structure and use of several embodiments of the invention as defined in the claims. Although various embodiments of the claimed invention have been described above with a certain degree of particularity, or with reference to one or more individual embodiments, other embodiments using different combinations of elements and structures disclosed herein are contemplated, as other iterations can be determined through ordinary skill based upon the teachings of the present disclosure. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A drainage container for use in a medical fluid drainage system, the drainage container comprising one or more walls that define a reservoir for holding a fluid therein, wherein one of the one or more walls includes a lid and a gas-permeable, liquid-impermeable membrane is positioned on a bottom surface of the lid;
an inlet port defined within one of the one or more walls and configured to be fluidly coupled to a drainage catheter;
an outlet port defined within one of the one or more walls and configured to be fluidly coupled to a drainage bag; and
a vacuum port defined within one of the one or more walls and configured to be fluidly coupled to a negative pressure source.

2. The drainage container of claim 1, wherein the lid forms a top surface of the drainage container and the vacuum port is defined within the lid.

3. The drainage container of claim 2, wherein the inlet port is further defined within the lid.

4. The drainage container of claim 2, wherein at least some of the one or more walls define an upper opening in the drainage container and the lid is removably attached to the drainage container to seat upon and cover the upper opening.

5. The drainage container of claim 2, wherein the lid is configured to form a fluid tight seal with edges of the one or more walls defining the upper opening.

6. The drainage container of claim 1, wherein the gas-permeable, liquid-impermeable membrane is further positioned to extend across a vent aperture located on the lid.

7. The drainage container of claim 1, further comprising a gas-permeable, liquid-impermeable membrane positioned to extend across a lumen of the vacuum port.

8. The drainage container of claim 7, wherein the gas-permeable, liquid-impermeable membrane is held in place by a retention plate affixed to the bottom surface of the lid.

9. The drainage container of claim 1, wherein the vacuum port includes an annular wall that extends beyond an outer surface of the one of the plurality of walls and is formed with a locking connection structure configured to connect with an opposing connector attached to the negative pressure source.

10. A medical fluid drainage system, comprising a drainage tube with a first end configured to be placed within a hollow organ or body cavity of a patient and a second end;
an evacuation tube with a first end configured to attach to a negative pressure source and a second end;
a transfer tube with a first end and a second end;
a drainage container that defines a reservoir for holding a fluid therein, wherein the drainage container includes a removable lid and a gas-permeable, liquid-impermeable membrane is positioned on a bottom surface of the removable lid;
an inlet port defined within a first portion of the drainage container and configured to be fluidly coupled to the second end of the drainage catheter;
an outlet port defined within a second portion of the drainage container and configured to be fluidly coupled to the first end of the transfer tube;
a vacuum port defined within a third portion of the drainage container and configured to be fluidly coupled to the second end of the evacuation tube; and
a drainage bag with an inlet port configured to be fluidly coupled to the second end of the transfer tube.

11. The medical fluid drainage system of claim 10, further comprising a negative pressure source coupled to the vacuum port via the evacuation tube and configured to pull air from the drainage container to maintain a pressure equilibrium within the drainage container and the drainage bag.

12. The medical fluid drainage system of claim 10, further comprising a positive pressure source coupled with the drainage tube and configured to provide air flow through the drainage tube and into the drainage container.

13. The medical fluid drainage system of claim 10, wherein the third portion of the drainage container defining the vacuum port forms a portion of the removable lid.

14. The medical fluid drainage system of claim 13, wherein the first portion of the drainage container defining the inlet port forms another portion of the removable lid.

15. The medical fluid drainage system of claim 10, wherein the gas-permeable, liquid-impermeable membrane is positioned to extend across a lumen of the vacuum port.

16. The medical fluid drainage system of claim 15, wherein a perimeter edge of the membrane is positioned between a retention plate and the bottom surface of the lid.

17. The medical fluid drainage system of any claim 10, wherein the lid further comprises a vent opening covered by the gas-permeable, liquid-impermeable membrane.

18. A method of providing medical fluid drainage to a patient, the method comprising receiving a bodily fluid from a patient into a drainage container through a drainage tube;
transferring at least a portion of the bodily fluid from the drainage container to a drainage bag fluidly connected to the drainage container;
removing excess air from and reducing pressure within the drainage container using a negative pressure source connected to the drainage container; and
removing excess air from the drainage bag via the fluid connection with the drainage container to provide an equilibrium pressure in the drainage bag.

19. The method of claim 18 further comprising introducing a positive pressure air flow into the drainage tube that flows into the drainage container.

20. The method of claim 18, wherein the drainage container comprises a removable lid defining a vacuum port connected to the negative pressure source and the step of removing excess air from the drainage container further comprises flowing air out of the drainage container through the vacuum port.
